(19) United States
(12) Patent Application Publication
(10) Pub. No.: US 2025/0018094 A1
(43) Pub. Date: Jan. 16, 2025

(54) HYDROGEL REINFORCEMENT USING EXPANDED ARTICLES AND HYDROGEL-EXPANDED ARTICLE COMPOSITES

(71) Applicant: W. L. Gore & Associates, Inc., Newark, DE (US)

(72) Inventors: Jared S. Archer, Flagstaff, AZ (US); Matthew I. Converse, Newark, DE (US); Alexander T. Leonard, Newark, DE (US); Jackson T. Lewis, Newark, DE (US); Theresa K. Tonge, Cumberland, ME (US)

(21) Appl. No.: 18/771,739

(22) Filed: Jul. 12, 2024

Related U.S. Application Data
(60) Provisional application No. 63/526,774, filed on Jul. 14, 2023.

(51) Int. Cl. A61L 31/14; A61L 31/12; C08J 9/42

(52) U.S. Cl. CPC A61L 31/145; A61L 31/129; C08J 9/42; C08J 2323/06; C08J 2327/18; C08J 2333/26; C08J 2363/10

(57) ABSTRACT

A reinforced hydrogel composite including a porous synthetic or naturally derived retracted membrane material having a void volume, and a hydrogel at least partially filling the void volume; wherein the composite has a low strain (<50%) modulus from about 0.01 to about 10 MPa and a toughness from about 10⁴ to about 10⁷ J·m³. Methods for making the reinforced hydrogel composite and articles containing the reinforced hydrogel composite are also provided.

### CROSS-REFERENCE TO RELATED APPLICATION

This application claims the benefit of Provisional Application No. 63/526,774, filed Jul. 14, 2023, which is incorporated herein by reference in its entirety for all purposes.

### FIELD

The present disclosure relates generally to hydrogels and methods for hydrogel reinforcement. More specifically, the disclosure relates to hydrogel-expanded article composites and methods for making and applying the same.

### BACKGROUND

Hydrogels have unique properties that make them desirable for a breadth of applications in the biomedical, agricultural, cosmetic, electronic, consumer, energy, and 3D printing industries. However, their weak mechanical properties can limit their adoption. Traditional efforts to improve their mechanics have consisted primarily of altering their underlying polymer architecture. While these efforts have resulted in radical improvements in mechanical performance, they often come at the cost of designing the polymer network for an adjacent property of interest such as cell response, cell seeding, transport properties, shape memory, or wear performance.

More recent work has focused on generating hydrogel composites. While these strategies have resulted in radical increases in strength, toughness, extensibility, and fracture resistance, the moduli of these composites are often several orders of magnitude higher than the neat hydrogel. In many of the applications that hydrogels are being considered, particularly in the biomedical space, preserving the low modulus of the neat hydrogel is critical.

### SUMMARY

The present disclosure provides a method for generating a hydrogel composite via a polymer fiber reinforcement that advantageously produces a composite with high toughness and strength while additionally achieving a low modulus at physiologically relevant strains.

According to one Example, the present disclosure provides a reinforced hydrogel composite including: a porous synthetic or naturally derived retracted membrane material having a void volume, and a hydrogel at least partially filling the void volume; wherein the composite has a low strain (<50%) modulus from about 0.01 to about 10 MPa and a toughness from about 10⁴ to about 10⁷ J·m³.

According to a second Example, the present disclosure provides a method of preparing a reinforced hydrogel composite including: i) providing a synthetic or naturally derived polymer membrane compressed in at least one direction, said polymer membrane having a node and fibril microstructure and a void volume; ii) optionally pre-treating the polymer membrane, iii) filling at least partially the void volume with a hydrogel precursor; and iv) polymerizing the hydrogel precursor to produce the reinforced hydrogel composite which includes a hydrogel embedded in the polymer membrane; wherein the composite has low strain (<50%) modulus from about 0.01 to about 10 MPa and a toughness of from about 10⁴ to about 10⁷ J·m³.

According to one embodiment (“Embodiment 1”), the disclosure relates to a reinforced hydrogel composite including: a porous synthetic or naturally derived retracted membrane material having a void volume, and a hydrogel at least partially filling the void volume; wherein the composite has a low strain (<50%) modulus from about 0.01 to about 10 MPa and a toughness from about 10⁴ to about 10⁷ J·m³.

Embodiment 2 is the reinforced hydrogel composite of Embodiment 1, wherein the membrane has a microstructure, optionally including serpentine fibrils or a node and fibril structure.

Embodiment 3 is the reinforced hydrogel composite of Embodiment 2, wherein the fibrils in the node and fibril microstructure have an average diameter from about 0.1 μm to 250 μm.

Embodiment 4 is the reinforced hydrogel composite of Embodiment 2, wherein the nodes in the node and fibril microstructure have an average separation distance of about 5 to 5000 μm.

Embodiment 5 is the reinforced hydrogel composite of any one of Embodiments 1 to 4, wherein the membrane includes macro-structured folds or micro-structured folded fibrils.

Embodiment 6 is the reinforced hydrogel composite of any one of Embodiments 1 to 5, wherein the membrane material is selected from the group consisting of expanded polytetrafluoroethylene (ePTFE), polyvinylidene fluoride (PVDF), fluorinated ethylene propylene (FEP), perfluoroalkoxy alkane (PFA), polyester sulfone (PES), expanded poly paraxylylene (ePPX), expanded ultra-high molecular weight polyethylene (eUHMWPE), expanded ethylene tetrafluoroethylene (eETFE), porous poly (tetramethyl-p-silphenylenesiloxane), expanded porous polylactic acid (ePLLA), polycaprolactone (PCL), polyurethane (PU), copolymers of polyglycolic acid (PGA) and trimethylene carbonate (TMC), silk fibroin, silk spidroin, cellulose, nanocellulose, polyhydroxyalkanoates, and any combination thereof.

Embodiment 7 is the reinforced hydrogel composite of any one of Embodiments 1 to 6, wherein the hydrogel is a polyampholyte hydrogel or a zwitterionic hydrogel.

Embodiment 8 is the reinforced hydrogel composite of Embodiment 7, wherein the polyampholyte hydrogel includes at least one cationic group and at least one anionic group.

Embodiment 9 is the reinforced hydrogel composite of Embodiment 8, wherein said cationic and anionic groups are randomly dispersed.

Embodiment 10 is the reinforced hydrogel composite of any one of Embodiments 1 to 9, wherein the hydrogel includes polyethylene glycol (PEG), polyvinyl alcohol (PVA), poly(2-hydroxyethyl methacrylate) (pHEMA), alginate, hyaluronic acid, or chitosan.

Embodiment 11 is the reinforced hydrogel composite of any one of Embodiments 1 to 10, wherein the hydrogel is cross-linked.

Embodiment 12 is the reinforced hydrogel composite of any one of Embodiments 1 to 11, wherein the hydrogel is cross-linked in situ.

Embodiment 13 is the reinforced hydrogel composite of any one of Embodiments 1 to 12, wherein the hydrogel completely fills the void volume.

Embodiment 14 is the reinforced hydrogel composite of any one of Embodiments 1 to 13, wherein the hydrogel further includes one or more neutral groups.

Embodiment 15 is the reinforced hydrogel composite of any one of Embodiments 1 to 14, wherein the reinforced hydrogel composite contains from 0.01 wt. % to 99 wt. % of the hydrogel based on the total weight of the reinforced hydrogel composite.

Embodiment 16 is the reinforced hydrogel composite of any one of Embodiments 1 to 15, further including at least one bioactive agent.

Embodiment 17 is the reinforced hydrogel composite of Embodiment 16, wherein the bioactive agent is selected from the group consisting of thrombo-resistant agents, antibiotic agents, anti-tumor agents, anti-viral agents, anti-angiogenic agents, angiogenic agents, anti-inflammatory agents, cell cycle regulating agents, and chemically modified equivalents and combinations thereof.

Embodiment 18 is the reinforced hydrogel composite of any one of Embodiments 1 to 17, wherein the polymer membrane is in the form of a tape, a sheet, a tube, a fiber, a filament, or a monolith.

Embodiment 19 is the reinforced hydrogel composite of Embodiment 18, wherein the tape, sheet, or tube has a thickness from about 1 μm to 5000 μm.

Embodiment 20 is the reinforced hydrogel composite of any one of Embodiments 1 to 19, wherein the hydrogel is covalently bonded to the polymer membrane.

Embodiment 21 is the reinforced hydrogel composite of any one of Embodiments 1 to 20, wherein the reinforced hydrogel composite contains from 10 to 95 volume percent of hydrogel within the void volume of the polymer membrane.

Embodiment 22 is a laminate including the reinforced hydrogel composite of any one of Embodiments 1 to 21.

Embodiment 23 is an article comprising the reinforced hydrogel composite of any one of Embodiments 1 to 21 or the laminate of Embodiment 22.

Embodiment 24 is the article of Embodiment 23, wherein the article is an implantable medical device, a stent, a sensor, a fuel cell, a garment, footwear, a cosmetic, or a filter.

Embodiment 25 is the article of Embodiment 24, wherein the implantable medical device is selected from the group consisting of conduits, vascular grafts, endovascular grafts, stents, graft-stents, catheters, guidewires, trocars, tissue scaffolds, and introducer sheaths.

Embodiment 26 is a method of preparing a reinforced hydrogel composite including:
i) providing a synthetic or naturally derived polymer membrane compressed in at least one direction, said polymer membrane having a node and fibril microstructure and a void volume; ii) optionally pre-treating the polymer membrane;
iii) filling at least partially the void volume with a hydrogel precursor; and
iv) polymerizing the hydrogel precursor to produce the reinforced hydrogel composite which comprises a hydrogel embedded in the polymer membrane;
wherein the composite has low strain (<50%) modulus from about 0.01 to about 10 MPa and a toughness of from about 10⁴ to about 10⁷ J·m³.

Embodiment 27 is the method of Embodiment 26, wherein the pre-treating of the polymer membrane comprises wetting the polymer membrane with a solvent.

Embodiment 28 is the method of Embodiment 27, wherein the solvent is isopropyl alcohol or acetone.

Embodiment 29 is the method of Embodiment 26, wherein the pre-treating of the polymer membrane includes wetting the polymer membrane with a hydrogel precursor solution.

Embodiment 30 is the method of Embodiment 29, wherein hydrogel precursor solution is UV-curable, chemically crosslinked, or physically crosslinked.

Embodiment 31 is the method of Embodiment 26, wherein the polymer membrane is a hydrophilic-treated membrane.

Embodiment 32 is the reinforced hydrogel composite of Embodiment 7, wherein the zwitterionic hydrogel includes poly(sulfobetaine methacrylate) (SBMA), poly(carboxybetaine methacrylate (CBMA), poly(2-methacryloyloxyethyl phosphorylcholine) (MPC), or carboxybetaine acrylamide (CBAA).

The foregoing Examples are just that and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a graphical illustration of stress-strain curves of the neat PEG gels (dashed lines) and the PTFE-reinforced PEG composites (solid lines). The right graph is a magnified version of the left. PTFE films were first subjected to an in-plane compression of either 0% (no compression), 17%, or 29%.

FIG. 2 is a graphic illustration of a log-log plot of modulus vs. toughness for the neat PEG gels (solid dots) and the PTFE-reinforced PEG composites (hollow shapes). While all composites were significantly tougher than the neat PEG gels, only composites with an in-plane-compressed reinforcement (hollow circles and squares) maintained a low-strain modulus comparable to that of the neat gel.

FIG. 3. is a graphical illustration of stress-strain curves of the neat PEG gels (dashed lines) and the PE-reinforced PEG composites (solid lines). The right graph is a magnified version of the left. PE films were first subjected to an in-plane compression of either 0% (no compression), or 29%.

FIG. 4. is a graphical illustration of a log-log plot of modulus vs. toughness for the neat PEG gels (solid dots) and the PE-reinforced PEG composites (hollow shapes). While all composites were significantly tougher than the neat PEG gels, only composites with an in-plane-compressed reinforcement (hollow circles) maintained a low-strain modulus comparable to that of the neat gel.

FIG. 5. is a graphical illustration of stress-strain curves of the neat PAAm gels (dashed lines) and the PTFE-reinforced PAAm composites (solid lines). The right graph is a magnified version of the left. PTFE films were first subjected to an in-plane compression of either 0% (no compression) or 23%.

FIG. 6. is a graphical illustration of a log-log plot of modulus vs. toughness for the neat PAAm gels (solid dots) and the PTFE-reinforced PAAm composites (hollow shapes). While all composites were significantly tougher than the neat PAAm gels, only composites with an in-plane-compressed reinforcement (hollow circles) maintained a low-strain modulus comparable to that of the neat gel.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

As used herein, the terms “compacted”, “retracted” or “structured” may be used interchangeably when referring to a polymeric reinforcement membrane that is compacted in one or more of the transverse direction or machine direction prior to the application of the hydrogel material. It is to be understood that the compaction is not limited to one direction: it may be done in the transverse direction, machine direction, or in both directions, either sequentially or simultaneously. In one exemplary embodiment, the polymeric reinforcement membrane is compacted in the transverse direction prior to the application of the hydrogel material. The polymeric reinforcement membrane subjected to compaction forms macro-structured folds and/or micro-folded fibrils in the membrane, giving the polymeric reinforcement membrane low modulus and flexibility. The compacted polymeric reinforcement membrane may also or alternatively demonstrate out-of-plane geometries such as wrinkles or folds in the membrane. It is to be noted that heat shrinkage or solvent shrinkage or other suitable method may alternatively be used to non-mechanically “compact” the polymeric reinforcement membrane.

As used herein, a “hydrogel” is defined as a three-dimensional polymer (natural or synthetic) network structure able to imbibe and retain large amounts of water, in most cases greater than 50%. Hydrogels do not typically dissolve due to chemical or physical cross-links and/or chain entanglements, however, they could be designed such that they dissolve with time. The cross-linking may be through primary covalent cross-links, ionic forces, hydrogen bonds, affinity or bio-recognition interactions, hydrophobic interactions, polymer crystallites, physical entanglements of individual polymer chains, a combination of two or more of the above interactions. The hydrogel may consist of a natural polymer (such as collagen, hyaluronic acid, chitosan, heparin, alginate, fibrin, agarose, methylcellulose, hyaluronan, elastin-like polypeptides, to name a few) or a synthetic polymer (such as polyvinyl alcohol, polyethylene glycol, sodium polyacrylate, acrylate polymers and copolymers thereof).

It is to be understood that throughout the application, the term “PTFE” is utilized herein for convenience and is meant to include not only polytetrafluoroethylene, but also expanded PTFE, expanded modified PTFE, and expanded copolymers of PTFE, such as described in U.S. Pat. No. 5,708,044 to Branca, U.S. Pat. No. 6,541,589 to Baillie, U.S. Pat. No. 7,531,611 to Sabol et al., U.S. Pat. No. 8,637,144 to Ford, and U.S. Pat. No. 9,139,669 to Xu et al.

### DESCRIPTION OF VARIOUS EMBODIMENTS

### I. Hydrogels

The present disclosure provides reinforced hydrogel composites which include a membrane material and a hydrogel filled within the membrane.

A hydrogel is a three-dimensional network of hydrophilic polymers of polymer chains that are capable of absorbing and retaining water or aqueous solutions. Hydrogels are typically formed through a crosslinking process, where polymer chains are chemically linked together to create a network structure. The crosslinking can be achieved through covalent bonding, physical entanglements, or a combination of both.

Hydrogels may be used in a variety of applications and are commonly used in biomedical applications, such as implantable articles. Other applications for hydrogels include tissue engineering scaffolds (cartilage, connective tissue, cardiovascular, neuron regeneration, reconstructive surgery.), agriculture (seaweed, almond, film farming, water supply, root systems), drug delivery, filtration and separations (chromatography, water purification, carbon dioxide capture, aqueous reaction separations, dye and heavy ion removal), soft robotics (synthetic muscle, surgery), energy storage (supercapacitors, batteries anode, separator, lithium ion conductors), wound dressings, and synthetic tissue replacement.

Use in implantable applications typically is associated with matching the mechanical properties of the implantable article to the properties of the surrounding tissue. Many hydrogels lack sufficient strength or toughness for the target application. As such, many applications may require combining a reinforcement matrix with the hydrogel to achieve higher mechanical durability.

### II. Polymeric Reinforcement Membranes and Films

A polymeric reinforcement matrix is used to create composite hydrogel articles with improved mechanical properties. The polymeric reinforcement membrane is a microporous membrane that may be prepared from a variety of polymers. In one embodiment, the polymeric reinforcement membrane includes a non-woven fibrous microstructure formed by traditional spinning techniques (melt spinning, solvent-based spinning (such as dry, wet, gel, electrospun, etc.) that may be combined with subsequent processing steps such as calendaring, drawing/orientation, and thermal conditioning. In another embodiment, the polymeric reinforcement membrane is formed by expanding/stretching (e.g., uniaxial, biaxial, radial) fibrillatable polymers. Non-wovens obtained typically have polymeric fibers/fibrils diameters ranging from nanometers to micrometers.

In an exemplary embodiment, the polymeric reinforcement membrane may be formed from a fibrillatable polymer by expanding/stretching a preform into a porous article having a fibrillated microstructure. The fibrillated microstructure may include nodes interconnected by fibrils. Alternatively, the membrane may contain macro-structured folds or micro-structured folded fibrils.

Specific examples of fibrillatable polymers (e.g., capable of being processed into a membrane having a node and fibril microstructure) include, but are not limited to ultrahigh molecular weight polyethylene (UHMWPE), polylactic acid, copolymers of vinylidene fluoride with tetrafluoroethylene or trifluoroethylene (e.g. VDF-co-(TFE or TrFE) polymers), poly (ethylene tetrafluoroethylene) (ETFE), polyparaxylxylene (PPX), polytetrafluoroethylene (PTFE), and (tetramethyl-p-silphenylenesiloxane).

In another embodiment, the polymeric reinforcement membrane is a porous fluoropolymer membrane. In at least one exemplary embodiment, the polymeric reinforcement membrane is a polytetrafluoroethylene (PTFE) membrane or an expanded polytetrafluoroethylene (ePTFE) membrane. In a preferred embodiment, the polymeric reinforcement membrane is an expanded polytetrafluoroethylene membrane. Expanded polytetrafluoroethylene (ePTFE) membranes prepared in accordance with several different methods including compression molding, ram extrusion, paste extrusion, gel spinning, suspension coagulation, solvent-induced phase separation, biaxial stretching, breathable film lamination, and sintering. Suitable methods also include processing polytetrafluoroethylene polymers below the melt temperature of the polytetrafluoroethylene polymer. Preferably, the expanded polytetrafluoroethylene (ePTFE) membranes prepared by the foregoing methods have high strength, a microstructure of nodes and fibrils, and high porosity.

Other suitable membrane materials include polyvinylidene fluoride (PVDF), fluorinated ethylene propylene (FEP), perfluoroalkoxy alkane (PFA), polyester sulfone (PES), expanded poly paraxylylene (ePPX), expanded ultra-high molecular weight polyethylene (eUHMWPE), expanded ethylene tetrafluoroethylene (eETFE), porous poly (tetramethyl-p-silphenylenesiloxane), expanded porous polylactic acid (ePLLA), polycaprolactone (PCL), polyurethane (PU), copolymers of polyglycolic acid (PGA) and trimethylene carbonate (TMC) (for example, GORE® BIO-A® available from W.L. Gore & Associates, as well as naturally-isolated or synthetically-produced biopolymers such as polypeptides/proteins (e.g. silk fibroins/spidroins), polyhydroxyalkanoate (PHA) homopolymers and copolymers, cellulose, nanocellulose, and combinations thereof.

The porous fluoropolymer membrane may also include a polymer material that includes a functional tetrafluoroethylene (TFE) copolymer material where the functional TFE copolymer material includes a functional copolymer of TFE and PSVE (perfluorosulfonyl vinyl ether), or TFE with another suitable functional monomer, such as, but not limited to, vinylidene fluoride (VDF), vinyl acetate, or vinyl alcohol. A functional TFE copolymer material may be prepared, for example, according to the methods described in U.S. Pat. No. 9,139,707 to Xu et al. or U.S. Pat. No. 8,658,707 to Xu et al.

In another embodiment, the porous reinforcement membrane having a node and fibril microstructure is formed from an ultra-high molecular weight polyethylene (UHMWPE). In a preferred embodiment, the porous reinforcement membrane is an expanded UHMWPE (“ePE”) having a fibrillated microstructure, preferably a microstructure of nodes interconnected by fibrils, produced by expanding/stretching an UHMWPE preform formed by a solventless process (solid state processing), a solvent-based process (gel processed UHWMPE), or by paste processing UHMWPE resin particles using a suitable hydrocarbon lubricant. In a preferred embodiment, the ePE porous reinforcement membrane is formed by paste-processing as described in U.S. Pat. No. 10,577,468 to Sbriglia. The ePE membrane/film may be formed from a homopolymer of ethylene or a copolymer of ethylene and at least one suitable comonomer including, but not limited to an alpha-olefin or cyclic olefin having 3-20 carbon atoms. Non-limiting examples of suitable comonomers include 1-butene, 1-pentene, 1-hexene, 1-heptene, 1-octene, cyclohexene, and dienes with up to 20 carbon atoms (e.g., butadiene, 1,4-cyclohexene). Comonomers may be present in the UHMWPE copolymer in an amount from about 0.001 mol % to about 10 mol %, from about 0.01 mo1 % to about 5 mol % or from about 0.001 mol % to about 1 mol %. The ePE membrane/film may also include a blend of a fibrillatable UHMWPE and one or more lower molecular weight polyethylene (e.g., having an average molecular weight below 1,000,000; such as HDPE).

In one embodiment, the fibrils in the node and fibril microstructure have an average diameter of as low as 0.01 μm, 0.1 μm, 1 μm, 5 μm, 10 μm, 25 μm, 50 μm, 75 μm, 100 μm, 125 μm, 150 μm, 175 μm, 200 μm, 225 μm, 250 μm, or within any range encompassed by any two of the foregoing values as endpoints. For example, the fibrils in the node and fibril microstructure have an average diameter from about 0.1 μm to 250 μm.

In one embodiment, the nodes in the node and fibril microstructure have an average separation distance of as low as 1 μm, 50 μm, 100 μm, 500 μm, 1000 μm, 1500 μm, 2000 μm, 2500 μm, 3000 μm, 3500 μm, 4000 μm, 4500 μm, 5000 μm, 5500 μm, 6000 μm, or within any range encompassed by any two of the foregoing values as endpoints.

In one embodiment, the polymeric reinforcement membrane has an areal density of 5 g/m² or less, 4 g/m² or less, 3 g/m² or less, 2 g/m² or less or 1 g/m² or less. In another embodiment, the polymeric reinforcement membrane has an areal density range of 5 g/m² to 0.01 g/m², 4 g/m² to 0.1 g/m², 3 g/m² to 0.1 g/m², 2 g/m² to 0.1 g/m², 4 g/m² to 0.2 g/m², or 3 g/m² to 0.2 g/m².

In another embodiment, the polymeric reinforcement membrane has a thickness of 1 cm or less, 0.5 cm or less, 0.1 cm or less, or 0.01 cm or less. In another embodiment, the polymeric reinforcement membrane has a thickness of at least 0.05 μm, at least 0.1 μm or at least 0.2 μm. In another embodiment, the polymeric reinforcement membrane has a thickness range of 0.5 cm to 0.05 μm, 5 mm to 0.05 μm, 1 mm to 0.05 μm, 500 μm to 0.05 μm, 100 μm to 0.05 μm, 50 μm to 0.05 μm, 40 μm to 0.05 μm, 30 μm to 0.05 μm, 20 μm to 0.05 μm, 15 μm to 0.05 μm, 40 μm to 0.1 μm, 30 μm to 0.1 μm, 20 μm to 0.1 μm, 20 μm to 0.1 μm, 15 μm to 0.1 μm, 10 μm to 0.1 μm, 40 μm to 0.2 μm, 30 μm to 0.2 μm, 20 μm to 0.2 μm or 10 μm to 0.2 μm.

The polymeric reinforcement membrane (prior to coating or imbibing) has a porosity of at least 10%, at least 20%, at least 30%, at least 40%, at least 50%, at least 60%, at least 70% or at least 80%. In one embodiment, the polymeric reinforcement membrane has a porosity range of 10% to 98%, 20% to 98%, 30% to 98%, 40% to 98%, 50% to 98%, 10% to 90%, 10% to 85%, 10% to 80%, 10% to 70%, or 20% to 85%.

The polymeric reinforcement membrane has a matrix tensile strength (MTS) prior to compaction in both the machine direction (MD) and the transverse direction (TD) (orthogonal to the MD) at least about 30 MPa, of at least about 50 MPa, of at least about 100 MPa, of at least about 150 MPa, of at least about 200 MPa or of at least 300 MPa. In terms of matrix tensile strength, the polymeric reinforcement membrane prior to compaction may be unbalanced (strength in one direction significantly different from the strength in the orthogonal direction) or balanced (strength in MD and TD similar). In another embodiment, the polymeric reinforcement membrane has a ratio of the matrix tensile strength determined as MD:TD from about 0.1:1.0 to 1.0:0.1, or from about 0.5:1.0 to 1.0:0.5, or from about 0.7:1.0 to 1.0:0.7.

In another embodiment, the polymer membrane is in the form of a tape, a sheet, a membrane/film, a tube, a fiber, a filament or a monolith.

In some embodiments, where the polymer membrane is in the form of a tape, sheet, or tube, the tape, sheet, or tube may have a thickness as low as 1 μm, 50 μm, 100 μm, 500 μm, 1000 μm, 1500 μm, 2000 μm, 2500 μm, 3000 μm, 3500 μm, 4000 μm, 4500 μm, 5000 μm, 5500 μm, 6000 μm, or within any range encompassed by any two of the foregoing values as endpoints. For example, the tape, sheet, or tube may have a thickness of from about 1 μm to 5000 μm.

### III. Structured or Compacted Reinforcement Membranes

The present disclosure also encompasses hydrogels with structured or compacted reinforcement membranes.

In some embodiments, the porous polymeric reinforcement membranes of Section II are subjected to structuring/compaction prior to the application of the hydrogel component. As used herein, the terms “structured” or “compacted” or “compressed” refers to a porous reinforcement film/membrane that has been subjected to compaction whereby the areal density of the porous film/membrane is increased (relative to the porous film prior to compaction) and the modulus is lowered by introducing micro-folded fibers/fibrils and/or macroscopic structures (wrinkles/folds) in the porous film/membrane. The relative amount of compaction/compression can be adjusted to selectively introduce only micro-folded fibrils (to minimize out-of-plane wrinkles/folds/buckles in the Z-axis) or may include a level of compaction capable of introducing out-of-plan geometries (in additional to micro-folder fibers/fibrils). The amount of compaction introduced is a percentage increase in areal density relative to the staring porous reinforcement membrane and may be adjusted to obtain the desired change in modulus. The compacted film/membrane remains sufficiently porous for coating/imbibing with the hydrogel (or forming the hydrogel within the compacted porous film in situ). In-plane compaction/structuring may be conducted uniaxially (in one direction), biaxially (in two orthogonal directions). In one embodiment, the in-plane compaction/compression is conducted in one direction (uniaxial). In another embodiment, the in-plane compaction/compression is conducted in at least two orthogonal directions (biaxial).

In one embodiment, the general method described in U.S. Pat. No. 11,097,527 to Zaggl et al. is used to form a compacted porous film/membrane such that there is little or no introduction of substantial macroscope structures in the z-axis (i.e., fiber/fibril compaction).

In another embodiment, the compacted polymeric reinforcement membrane may also or alternatively demonstrate out-of-plane geometries such as wrinkles or folds (“buckles”) in the membrane, such as, but not limited to, the methods described in EP3061598 A1 to Zaggl et al. and U.S. Pat. No. 9,849,629 to Zaggl, et al. It is to be noted that heat shrinkage or solvent shrinkage or other suitable method may alternatively be used to non-mechanically “compact” the polymeric reinforcement membrane.

The “buckles” or out-of-plane structures in the compacted film/membrane may have a height that is at least two times the thickness of the non-compacted film/membrane. In addition, the height of the out-of-plane (i.e., z-direction) structures may range from about 2 μm to about 2000 μm or from about 20 μm to about 1000 μm. Further, the structure density in at least one direction is at least 1 buckle per mm, at least 2 buckles per mm, at least 3 buckles per mm, at least 4 buckles per mm, at least 5 buckles per mm, at least 6 buckles per mm, at least 7 buckles per mm, at least 8 buckles per mm, at least 9 buckles per mm, or at least 10 buckles per mm. In some embodiments, the structure density is from 1 buckle per mm to 10 buckles per mm, from 1 buckle per mm to 7 buckles per mm, from 1 buckle per mm to 5 buckles per mm, or from 1 buckle per mm to 3 buckles per mm.

The relative amount of compaction/compression introduced in-plane into the porous polymeric reinforcement membrane may be adjusted to achieve the desired mechanical properties. The percentage of compression/compaction introduced into the porous reinforcement membrane/film ranges is at least 1%, 2%, 3%, 4%, 5%, 6%, 7%, 8%, 9%, or 10% (relative to the increase in areal density or porous reinforcement film/membrane prior to compression) in at least one direction. The maximum amount of compaction/compression is no more than 80%, 70%, 60%, 50%, 40% or 30% in at least one direction. In a further embodiment, the percentage of compaction/compression is 1% to 80%, 3% to 70%, 3% to 60%, 3% to 50%, 3% to 40%, 5% to 80%, 5% to 70%, 5% to 60%, 5% to 50%, 5% to 40%, 10% to 80%, 10% to 70%, 10% to 60%, 10% to 50% or 10% to 40% in at least one direction.

In some embodiments the retracted membrane material may be a layer of polymer composite, which may alternatively be nonporous, microporous, or porous. Various nonporous materials which may be used in a retracted membrane material may include polymer films (e.g., TPU, PET, Silicone, Polystyrene block copolymer, FEP, and the like) or polymer composites. Porous materials, which may include expanded polytetrafluoroethylene (ePTFE) materials and ePTFE composite materials, provide a good balance of acoustics and water protection. Various porous and nonporous materials have excellent acoustic transference and provide excellent water protection, in addition to being very thin and lightweight. In some cases, a membrane material can be treated. For example, a membrane material can include an oleophobic coating, e.g., by an oleophobic polymer, before or after the membrane material is retracted.

In various embodiments, the retracted membrane material has a microstructure including serpentine fibrils. As used herein, the term “serpentine fibrils” means multiple fibrils that curve or turn one way then another. The serpentine fibrils may generally have a width of about 1.0 micron or less. The serpentine fibrils may be connected by nodes. Serpentine fibrils may be formed by, e.g., controlled retraction as described in U.S. Patent Pub. No. 2013/0183515. Controlled retraction can be achieved by causing articles to shorten in length in at least one direction by the application of heat, by wetting with a solvent, or by any other suitable means or combinations thereof in such a way as to inhibit folding, pleating, or wrinkling of the subsequent article visible to the naked eye. A retracted membrane can be created by retracting a precursor membrane to convert a substantial portion of fibrils therein into serpentine fibrils. In some cases, articles that have been retracted in accordance with the teachings of the present disclosure may require elongation in the direction of retraction in order to identify the serpentine fibrils.

### IV. Reinforced Hydrogel Composites

The present disclosure provides hydrogel composites which have at least one hydrogel partially or fully imbibed into a compacted polymeric reinforcement membrane.

In one embodiment, the reinforced hydrogel composite includes a porous synthetic or naturally derived retracted membrane material having a void volume, and a hydrogel at least partially filling the void volume; wherein the composite has a low strain (<50%) modulus from about 0.01 to about 10 MPa and a toughness from about 10⁴ to about 10⁷ J·m³.

The reinforced hydrogel composite may have a low strain (<50%) modulus from as low as 0.01 MPa, 0.1 MPa, 1 MPa, 2 MPa, 3 MPa, 4 MPa, 5 MPa, 6 MPa, 7 MPa, 8 MPa, or as high as 9 MPa, 10 MPa, 11 MPa, 12 MPa, 13 MPa, 14 MPa, 15 MPa, or within any range encompassed by any two of the foregoing values as endpoints. For example, the reinforced hydrogel composite may have a low strain (<50%) modulus of from 0.01 MPa to 10 MPa.

The reinforced hydrogel composite may have a toughness of as low as 10¹ J·m³, 10² J·m³, 10³ J·m³, 10⁴ J·m³, 10⁵ J·m³, 10⁶ J·m³, 10⁷ J·m³, 10⁸ J·m³, 10⁹ J·m³, 10¹⁰ J·m³, or within any range encompassed by any two of the foregoing values as endpoints. For example, the reinforced hydrogel composite may have a toughness of from about 10⁴ J·m³ to about 10⁷ J·m³.

In some embodiments, the reinforced hydrogel composite includes 0.01 wt. % to 99 wt. % hydrogel (weight percent based on the total weight of the reinforced hydrogel composite). In some embodiments, the reinforced hydrogel composite includes at least 0.01 wt. %, 2 wt. %, 3 wt. %, 4 wt. %, 5 wt. %, 6 wt. %, 7 wt. %, 8 wt. %, 9 wt. %, 10 wt. %, 15 wt. %, 20 wt. %, 25 wt. %, 30 wt. %, 35 wt. %, 40 wt. %, 45 wt. %, 50 wt. %, 55 wt. %, 60 wt. %, 65 wt. %, 70 wt. %, 75 wt. %, 80 wt. %, 85 wt. %, 90 wt. %, 95 wt. %, 96 wt. %, 97 wt. %, 98 wt. %, or 99 wt. % hydrogel (weight percent based on the total weight of the reinforced hydrogel composite).

In some embodiments, the reinforced hydrogel composite contains as low as 0.01 wt. % of the hydrogel (weight based on the total weight of the reinforced hydrogel composite), 0.1 wt. %, 1 wt. %, 2 wt. %, 3 wt. %, 4 wt. %, 5 wt. %, 6 wt. %, 7 wt. %, 8 wt. %, 9 wt. %, 10 wt. %, 15 wt. %, 20 wt. %, 25 wt. %, 30 wt. %, 35 wt. %, 40 wt. %, 45 wt. %, 50 wt. %, or as high as 55 wt. %, 60 wt. %, 65 wt. %, 70 wt. %, 75 wt. %, 80 wt. %, 85 wt. %, 90 wt. %, 95 wt. %, 96 wt. %, 97 wt. %, 98 wt. %, or 99 wt. %, or within any range encompassed by any two of the foregoing values as endpoints. For example, the reinforced hydrogel composite may contain from 0.01 wt. % to 99 wt. % of the hydrogel, based on the total weight of the reinforced hydrogel composite.

In another embodiment, the reinforced hydrogel composite includes 1 wt. % to 99 wt. % porous synthetic or naturally derived retracted membrane material (weight percent based on the total weight of the reinforced hydrogel composite). In one embodiment, the reinforced hydrogel composite includes at least 1 wt. %, 2 wt. %, 3 wt. %, 4 wt. %, 5 wt. %, 6 wt. %, 7 wt. %, 8 wt. %, 9 wt. %, 10 wt. %, 15 wt. %, 20 wt. %, 25 wt. %, 30 wt. %, 35 wt. %, 40 wt. %, 45 wt. %, 50 wt. %, 55 wt. %, 60 wt. %, 65 wt. %, 70 wt. %, 75 wt. %, 80 wt. %, 85 wt. %, 90 wt. %, 95 wt. %, 96 wt. %, 97 wt. %, 98 wt. %, or 99 wt. % of the porous synthetic or naturally derived retracted membrane material (weight percent based on the total weight of the reinforced hydrogel composite).

The hydrogel component partially or completely fills the void volume of the porous synthetic or naturally derived retracted membrane material. In one embodiment, the hydrogel includes at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the void volume in the compacted reinforcement membrane. In another embodiment, essentially 100% of the void volume in membrane is filled with the hydrogel. In another embodiment, the hydrogel completely fills the void volume.

The reinforced hydrogel composite may contain as low as 10 volume percent of the hydrogel within the void volume of the polymer membrane, or as low as 20 volume percent, 30 volume percent, 40 volume percent, 50 volume percent, 60 volume percent, or as high as 70 volume percent, 80 volume percent, 90 volume percent, 95 volume percent, or within any range encompassed by any two of the foregoing values as endpoints. For example, the reinforced hydrogel composite may contain from 10 to 95 volume percent of hydrogel within the void volume of the polymer membrane.

The hydrogel may be crosslinked and then imbibed into the membrane/film or the hydrogel may be formed in situ (formed from hydrogel precursors imbibed into the compacted reinforcement membrane). In a preferred embodiment, the hydrogel is formed from polymerizing or crosslinking hydrogel precursors imbibed within the compacted reinforcement membrane.

In some embodiments, the hydrogel is covalently bonded to the polymer membrane.

The reinforced hydrogel composite has a thickness of 1 cm or less, 0.5 cm or less, 0.1 cm or less, or 0.01 cm or less. In another embodiment, the polymeric reinforcement membrane has a thickness of at least 0.05 μm, at least 0.1 μm or at least 0.2 μm. In another embodiment, the polymeric reinforcement membrane has a thickness range of 0.5 cm to 0.05 μm, 5 mm to 0.05 μm, 1 mm to 0.05 μm, 500 μm to 0.05 μm, 100 μm to 0.05 μm, 50 μm to 0.05 μm, 40 μm to 0.05 μm, 30 μm to 0.05 μm, 20 μm to 0.05 μm, 15 μm to 0.05 μm, 40 μm to 0.1 μm, 30 μm to 0.1 μm, 20 μm to 0.1 μm, 20 μm to 0.1 μm, 15 μm to 0.1 μm, 10 μm to 0.1 μm, 40 μm to 0.2 μm, 30 μm to 0.2 μm, 20 μm to 0.2 μm or 10 μm to 0.2 μm.

In another embodiment, at 5% stress the reinforced hydrogel composite (including the compacted reinforcement membrane) has a reduced strain relative to a reinforced hydrogel composite having a reinforcement membrane that has not been compacted (i.e., 0% compaction). In one embodiment, at 5% stress the reinforced hydrogel composite has a strain greater than the unreinforced hydrogel. In another embodiment, at 5% stress the reinforced hydrogel composite has a strain greater than the unreinforced hydrogel (for example, greater than 2.5 kPa for unreinforced PEG) and a strain of less than 500 kPa, less than 250 kPa, less than 200 kPa, less than 100 kPa, less than 50 kPa or less than 25 kPa.

The components may include viscous chemical compositions, such as, but not limited to, a hydrogel material. Biologically active substances may optionally be combined with a hydrogel material or with any other added chemical component. With hydrogel materials, for example, the biologically active substances may be released directly from the hydrogel material or they may be released as the hydrogel material and the underlying expanded material are absorbed by the body of an implant recipient.

Suitable hydrogel can be made from various natural or synthetic polymers. The hydrogels encompassed by the present disclosure may include or be derived from polyvinyl alcohol, polyethylene glycol, polypropylene glycol, dextran, agarose, alginate, carboxymethylcellulose, hyaluronic acid, polyacrylamide, polyglycidol, poly(vinyl alcohol-co-ethylene), poly(ethylene glycol-co-propylene glycol), poly(vinyl acetate-co-vinyl alcohol), poly(tetrafluoroethylene-co-vinyl alcohol), poly(acrylonitrile-co-acrylamide), poly(acrylonitrile-co-acrylic acid-acrylamidine), poly(acrylonitrile-co-acrylic acid-co-acrylamidine), polyacrylic acid, poly-lysine, polyethyleneimine, polyvinyl pyrrolidone, polyhydroxyethylmethacrylate, poly(2-hydroxyethyl methacrylate) alginate, hyaluronic acid, or chitosan, polysulfone, mercaptosilane, aminosilane, hydroxylsilane, polyallylamine, polyaminoethylmethacrylate, polyomithine, polyaminoacrylamide, polyacrolein, acryloxysuccinimide, or their copolymers, either alone or in combination. Suitable solvents for dissolving the hydrophilic polymers include, but are not limited to, water, alcohols, dioxane, dimethylformamide, tetrahydrofuran, and acetonitrile, etc.

The hydrogels of the present disclosure may also be polyampholyte hydrogels. These polyampholyte hydrogels may include at least one cationic group and at least one anionic group which may also be randomly dispersed throughout the hydrogel (for example, see Ihsan et al., Macromolecules (2016) 49:4245-4252 and Sun et al., Nat. Mat. (2013) 12:932-937). In another embodiment, the hydrogels may be zwitterionic hydrogels such as poly(sulfobetaine methacrylate) (SBMA), poly(carboxybetaine methacrylate (CBMA), poly(2-methacryloyloxyethyl phosphorylcholine) (MPC), and carboxybetaine acrylamide (CBAA) (Carr et al., Biomaterials (2011) 32: 6893e6899 and Zhang et al., Carbohydrate Polymers (2021) 257:117627). In another embodiment, the hydrogel may further include one or more neutral groups.

Optionally, the compositions can be chemically altered after being combined with the expanded PLA polymer. These chemical alterations can be chemically reactive groups that interact with polymeric constituents of the expanded PLA polymer or with chemically reactive groups on the compositions themselves. The chemical alterations to these compositions can serve as attachment sites for chemically bonding yet other chemical compositions, such as biologically active substances. “Bioactive substances” include enzymes, organic catalysts, ribozymes, organometallics, proteins, glycoproteins, peptides, polyamino acids, antibodies, nucleic acids, steroidal molecules, antibiotics, antimycotics, cytokines, carbohydrates, oleophobics, lipids, extracellular matrix material and/or its individual components, pharmaceuticals, and therapeutics. One non-limiting example of a chemically based bioactive substance is dexamethasone. Cells, such as, mammalian cells, reptilian cells, amphibian cells, avian cells, insect cells, planktonic cells, cells from non-mammalian marine vertebrates and invertebrates, plant cells, microbial cells, protists, genetically engineered cells, and organelles, such as mitochondria, are also bioactive substances. In addition, non-cellular biological entities, such as viruses, virenos, and prions are considered bioactive substances herein.

### V. Additional Materials

The hydrogel composites of the present disclosure may also include additional materials such as organic and/or inorganic particular fillers, additional polymers, and bioactive agents. In one embodiment, the additional materials may include materials to improve friction mechanics, enhance anti-fouling performance, and the like.

In one embodiment, the bioactive agents such as thrombo-resistant agents, antibiotic agents, anti-tumor agents, anti-viral agents, anti-angiogenic agents, angiogenic agents, anti-inflammatory agents, cell cycle regulating agents, and chemically modified equivalents and combinations thereof.

The reinforced hydrogel composite may include up to 90 wt. % of the additional materials (based on the total weight of the reinforced hydrogel composite). In one embodiment, reinforced hydrogel composite may include 0.1 wt. % to 90 wt. %, 0.1 wt. % to 80 wt. %, 0.1 wt. % to 70 wt. %, 0.1 wt. % to 60 wt. %, 0.1 wt. % to 50 wt. %, 0.1 wt. % to 40 wt. %, 0.1 wt. % to 30 wt. %, 0.1 wt. % to 25 wt. %, 0.1 wt. % to 20 wt. %, 0.1 wt. % to 15 wt. % or 0.1 wt. % to 10 wt. % of the additional material(s).

### VI. Laminates

The present disclosure also provides a laminate including the reinforced hydrogel composite described in Section IV above. The reinforced hydrogel composite may be laminated to one or more woven or non-woven support materials.

### VII. Articles Including the Reinforced Hydrogel Composites

The present disclosure also provides an article containing the reinforced hydrogel composite or laminate described in Sections IV and VI above.

The article may be an implantable medical device, a stent, a sensor, a fuel cell, a garment, footwear, a cosmetic, or a filter.

Suitable medical devices include conduits, vascular grafts, endovascular grafts, stents, graft-stents, catheters, guidewires, trocars, tissue scaffolds, and introducer sheaths.

### VIII. Methods for Preparing Reinforced Hydrogel Composites

The present disclosure also provides methods for preparing the reinforced hydrogel composites described herein. The method may include the following steps:
i) providing a synthetic or naturally derived polymer membrane compressed in at least one direction, said polymer membrane having a node and fibril microstructure and a void volume;
ii) optionally pre-treating the polymer membrane;
iii) filling at least partially the void volume with a hydrogel precursor; and
iv) polymerizing the hydrogel precursor to produce the reinforced hydrogel composite which includes a hydrogel embedded in the polymer membrane;
   wherein the composite has low strain (<50%) modulus from about 0.01 to about 10 MPa and a toughness of from about 10⁴ to about 10⁷ J·m³.

In some embodiments, the pre-treating of the polymer membrane may involve wetting the polymer membrane with a solvent or solvent mixture that provides sufficient polymer membrane wetting as well as hydrogel precursor solubility. Examples of suitable solvents include isopropyl alcohol, acetone or mixtures thereof.

In some embodiments, the pre-treating of the polymer membrane may involve wetting the polymer membrane with a hydrogel precursor solution. Suitable hydrogel precursor solutions include UV-curable solutions, chemically cross-linkable solutions, or physically cross-linkable solutions.

In another embodiment, the optional pre-treating step is omitted, and the polymer membrane may be a membrane that is already treated such as a hydrophilic treated membrane.

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale but may be exaggerated to illustrate various aspects of the present disclosure, and in that regard, the drawing figures should not be construed as limiting.

### TEST METHODS

It should be understood that although certain methods and equipment are described below, other methods or equipment determined suitable by one of ordinary skill in the art may be alternatively utilized.

A variety of test methods may be used to characterize the reinforcing membranes and composite materials (such as measuring the porosity, thickness, and mechanical properties).

### Mass Per Area (Mass/Area)

The mass per area of samples is measured according to the ASTM D 3776 (Standard Test Methods for Mass Per Unit Area (Weight) of Fabric) test method (Option C) using a Mettler-Toledo Scale, Model 1060. The scale is recalibrated prior to weighing specimens, and the results are reported in grams per square meter (g/m²).

### Thickness

Membrane thickness is measured by placing the sample (e.g. composite membrane) between the two plates of a Kafer FZ1000/30 thickness snap gauge (Käfer Messuhrenfabrik GmbH, Villingen-Schwenningen, Germany). The average of the three measurements is used.

### Bulk Density

The bulk density of the sample is the density of the solid including all open pores and internal void volume. The bulk density is calculated by dividing the sample mass by its low-pressure mercury intrusion volume. Sample mass is determined by weighing on an analytical balance of +/− 0.01 mg sensitivity.

Bulk Density=M/(V_{Low Pressure})

### Mercury Porosimetry Testing

Porosity measurements may be conducted on a Micromeritics AutoPore V mercury porosimeter (Micromeritics, Norcross, Ga., USA), using Micromeritics MicroActive software version 2.0. Quadruple Distilled Virgin Mercury—99.9995% purity (Bethlehem Apparatus, Bethlehem, PA) is used as received for tests. Tests may use a solid type of penetrometer with a bulb volume of 5 cc and a stem volume of 0.392 cc (SN: 07-0979). Pieces of the composite samples are cut into 1 cm×2 cm strips and enough of these strips are weighed on an analytical balance to provide a total mass of approximately 0.25 g. After noting the mass, the sample pieces are placed in the penetrometer.

The test parameters may be as follows: (1) the penetrometer is placed into the low pressure port on the AutoPore and evacuated to 50 μm Hg, followed by 5 min unrestricted evacuation; (2) the penetrometer is then filled with mercury at 0.5 psia (˜3.5 kPa) and equilibrated for 10 seconds; pressure is subsequently applied to the capillary using nitrogen in steps up to 30 psia (˜0.21 MPa), equilibrating for 10 seconds at each step prior to determining the intrusion volume via the standard capacitance measurement with the penetrometer capillary; (3) the penetrometer is then removed from the low pressure port after returning to atmospheric pressure and then weighed to determine the amount of mercury added; (4) the penetrometer is subsequently placed into the high pressure port on the AutoPore and the pressure is again increased in a series of steps up to approximately 60,000 psia (˜413.7 MPa) allowing 10 sec at each step to equilibrate prior to intrusion volume measurements.

The intrusion volume V at any pressure is determined through a capacitance measurement using the pre-calibrated capillary (i.e., a cylindrical capacitor where the outer contact is the metallized coating on the external surface of the glass capillary, the inner contact is the liquid mercury, and the dielectric is the glass capillary). The total intrusion volume divided by the sample mass gives the specific intrusion volume (in mL/g).

The volume occupied by the sample is then calculated at the two extreme target pressures, namely, 0.5 psia (˜3.5 kPa) and 60,000 psia (˜413.7 MPa). Since the penetrometer has a known calibrated volume, the difference between this volume and the mercury volume (determined from the mass increase after mercury addition at low pressure and the density of mercury) yields the volume of the sample including any pores. Dividing the mass of the sample by the volume at this low pressure provides the bulk density of the sample. At high pressure, where mercury has been pushed into the pores by an amount given by the intrusion volume, the skeletal density can be approximated by dividing the sample mass by the adjusted sample volume (e.g., low pressure volume minus total intrusion volume).

### Skeletal Density

The skeletal density is the density of a solid calculated by excluding all open pores and internal void volume. The skeletal density is calculated by dividing the sample mass by the adjusted sample volume (low pressure volume minus total intrusion volume). The sample mass is determined by weighing on an analytical balance of +/− 0.01 mg sensitivity. A formula for skeletal density is provided below:

Skeletal Density=M/((V_{Low Pressure})−(V_{High Pressure}))

where V_{Low Pressure} is volume of the sample at 0.5 psia (˜3.5 kPa) and V_{High Pressure} is total intrusion volume at 60,000 psia (˜413.7 MPa).

### Total Porosity

The total porosity within the substrate is simply the void volume of the sample divided by the total volume of the sample. This can be calculated as: % Porosity=100*(total intrusion volume at 60,000 psia (˜413.7 MPa))/(volume of the sample at 0.5 psia (˜3.5 kPa)).

### Tensile Properties

Tensile properties can be measured on an Instron tensile tester based on the ASTM standard D412F. The tensile specimens are formed into dog bone shaped with total length of 12.70 cm (5.0 in) and width of 0.64 cm (0.25 in). The gauge length is 5.89 cm (2.32 in) and crosshead speed is 47.12 cm/min (18.55 in/min). Three measurements can be made for both MD and TD. The break load is then calculated for each direction as the average of the three measurements.

### EXAMPLES

Unless defined otherwise herein, all technical and scientific terms used herein have the same meaning as commonly understood by one of ordinary skill in the art to which this invention belongs. The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

Unless otherwise noted, materials used in hydrogel preparation and solvents were obtain from Sigma-Aldrich, St. Louis, MO. Polytetrafluoroethylene films were prepared by W.L. Gore & Associates, Newark, DE.

### Example 1

This first example demonstrates the use of an in-plane-compressed polytetrafluoroethylene (PTFE) film to mechanically reinforce a polyethylene glycol (PEG) hydrogel without substantially altering the modulus of the gel. Two levels of in-plane film compression (17% and 29%) are shown relative to a comparative example where the film was not compressed (0%).

### Uniaxial In-Plane Compression of Porous Reinforcing Film

A porous film of expanded PTFE (ePTFE) prepared according to the methodology in U.S. Pat. No. 5,814,405 to Branca et al. was subjected to a uniaxial in-plane compression with the aid of a silicone carrier using methodology described in U.S. Pat. No. 11,097,527 to Zaggl et al. Briefly, a rectangular sheet of 0.4 mm thick silicone with a durometer of 27 Shore A was secured in a lab machine with roughly a half dozen clamps on each of the four sides. Using the software-controlled clamps, the silicone was uniformly stretched such that one dimension of the rectangular sheet was elongated to a target stretch ratio, A. The reinforcing film was then laid flat on the silicone sheet with the desired compression axis parallel to the silicone sheet's axis of deformation. Any residual air pockets between the film and silicone were removed with the aid of a vacuum plate. The clamps were then returned to their original position, effectively restoring the silicone to its undeformed state while simultaneously subjecting the film to an in-plane uniaxial compression of magnitude 1−1/λ.

TABLE 1
Properties of the porous ePTFE reinforcing film

| Measurement | Value |
|---|---|
| Mass per area (g/m²), measured | 1.43 |
| Non-contact thickness (μm), measured | 7.96 |
| Porosity (%), calculated | 91.9 |
| Primary axis strength per width (N/mm) (MD) | 0.64 |
| Secondary axis strength per width (N/mm) (TD) | 0.1 |
| Ratio of orthogonal strengths (MD:TD) | 6.2 |

### Removal of Compressed Film From Silicone Carrier

Following in-plane compression, small regions of the film were individually removed from the silicone with the aid of a flexible, adhesive-backed plastic frame. Briefly, an adhesive-backed plastic sheet was cut into roughly 40 mm squares and the center of each was punched out with a 25×19 mm die. The frame was then adhered to the compressed film with its 25 mm length aligned with the axis of film compression. A blade was used to cut the film around the frame and the frame (with the film now attached) was peeled away from the silicone by curling the frame about the axis of film compression to mitigate stretching in that direction. Framed films were then set aside for later imbibing with the hydrogel precursor solution or for testing as neat films.

### Hydrogel Precursor Solution

A precursor solution of polyethylene glycol diacrylate (PEG-DA), Product #455008, CAS-No: 26570-48-9 Sigma-Aldrich, St. Louis, MO 63103) was formulated for subsequent curing as either a neat hydrogel or as a reinforced hydrogel composite. PEG-DA with an average Mₙ=700 and 2-Hydroxy-4′-(2-hydroxyethoxy)-2-methylpropiophenone (CAS 106797-53-9; MW 224.25; IRGACURE® 2959; PubChem Substance ID: 24865712, Product #410896; Sigma-Aldrich, St. Louis, MO 62103) used as received. The precursor was prepared using PEG-DA (10 wt. %) and 0.075 wt. % IRGACURE® 2959 dissolved in a solvent mixture of deionized (DI) water (50 wt. %), isopropyl alcohol (IPA) (30 wt. %), and n-butanol (20 wt. %). This precursor was made in advance and stored at 4° C. until the day it was injected and cured.

### Imbibing and Curing of the Hydrogel Precursor to Create Reinforced Gel Composites

Hollow, air-tight wells were formed between clean sheets of glass by sandwiching a 1 mm thick perimeter of silicone. The previously framed film was placed within the well and the precursor was injected through the silicone perimeter with a hypodermic needle. Composites were then cured under a broad-spectrum UV lamp (Loctite 7411-S UV Flood System) for at least 5 mins and subsequently immersed in DI water to exchange with the butanol and IPA.

### Uniaxial Tensile Tests

The uniaxial stress-strain behavior of neat gels and composites was characterized on a TA Instruments ELECTROFORCE® 3200 Dynamic Mechanical Analyzer (DMA) (TA Instruments-Waters LLC, New Castle, Delaware) with an extended stroke actuator and 22 N load cell. Samples were cut to a 10 mm width and gripped to a 25 mm gauge length. Composites were oriented such that the axis of the reinforcing film's prior in-plane compression was aligned with the loading direction. In the case of composites, the layer of unreinforced gel (as the gel was thicker than the reinforcement) was removed in the region of the grips to enable a strong grip for loading to relatively high forces. In the case of neat gels, two pieces of sandpaper were glued to the front and back faces of the gel at each end to facilitate gripping without crushing the gel. All samples were pulled to failure at a rate of 10%/s. Time, displacement, and force signals were collected at 100 Hz with a 30 Hz digital filter applied to the force signal. Stress was calculated as the force divided by the sample cross-sectional area (10 mm width and approx. 1 mm thick). Modulus was measured by fitting a line to the stress-strain curve spanning 0-2% strain. Toughness was calculated as the area under the stress-strain curve up to failure. Tabulated values are reported as mean±standard deviation.

### Results

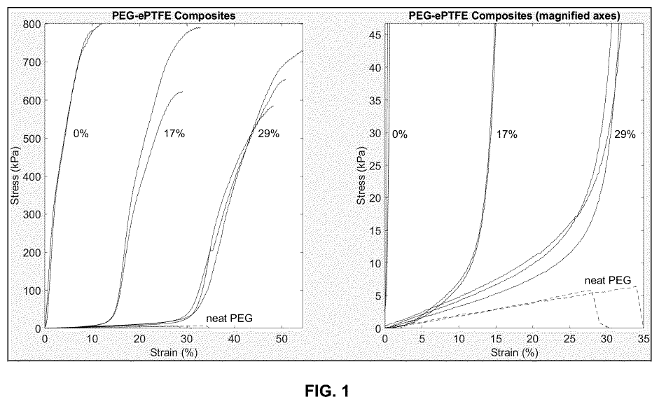

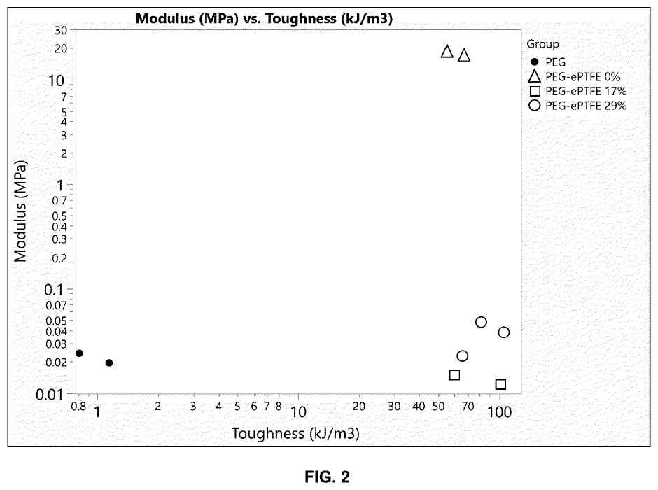

Analysis of the stress-strain curves (FIG. 1) and quantification of them (FIG. 2) demonstrated that while all composites were significantly stronger and tougher than the unreinforced gel, only composites with an in-plane-compressed reinforcement had a low-strain modulus comparable to that of the gel. Mean (±std) values of the modulus and toughness graphically illustrated in FIG. 2 are provided in Table 2

TABLE 2
Silicone stretch ratio, resulting degree of in-plane film compression,
and mechanical properties for the composites and the neat PEG gel

| | | | In-plane | | |
|---|---|---|---|---|---|
| | | Silicone | Compression | | |
| | | Stretch | (1 − 1/λ) | Modulus | Toughness |
| Sample ID | Description | Ratio, λ | (%) | (kPa) | (kJ/m³) |
| 1 | PEG-ePTFE | 1.0 (no | 0% (no | 18,000 ± 1,100 | 61 ± 8.4 |
| (comparative) | 0% | stretch) | compression) | | |
| 2 | PEG-ePTFE | 1.2 | 17% | 14 ± 2.0 | 81 ± 30 |
| | 17% | | | | |
| 3 | PEG-ePTFE | 1.4 | 29% | 36 ± 13 | 84 ± 20 |
| | 29% | | | | |
| 4 | Neat PEG gel | N/A | N/A | 22 ± 3.3 | 0.97 ± 0.23 |

Furthermore, Example 1 demonstrates the relatively sharp transition which can be achieved between the low-and high-modulus regimes. In some applications it may be desirable for the strain-stiffening to occur before reaching a defined strain. This sharp transition may make it possible to do so while still maximizing the strain window over which the initial modulus is low.

### Example 2

The methodology of Example 1 was used except that the reinforcing membrane is made out of Ultra-High Molecular Weight Polyethylene (UHMWPE) prepared following the method described in U.S. Pat. No. 10,266,670 to Sbriglia. Properties of the ePE film are provided in Table 3. We again reinforce a PEG hydrogel and do so without substantially altering the modulus of the gel. One level of in-plane film compression is used (29%) and compared relative to the case where the reinforcing film is not compressed (0%) prior to imbibing and curing the hydrogel.

TABLE 3
Properties of the porous PE reinforcing film

| Measurement | Value |
|---|---|
| Mass per area (g/m²), measured | 2.35 |
| Non-contact thickness (μm), measured | 9 |
| Porosity (%), calculated | 72.5 |
| Primary axis strength per width (N/mm) | 0.75 |
| (MD) | |
| Secondary axis strength per width (N/mm) | 0.69 |
| (TD) | |
| Ratio of orthogonal strengths | 1.1 |

Film compression and handling, gel imbibing and curing, and the subsequent mechanical characterization were all conducted and analyzed in a manner identical to that outlined in Example 1.

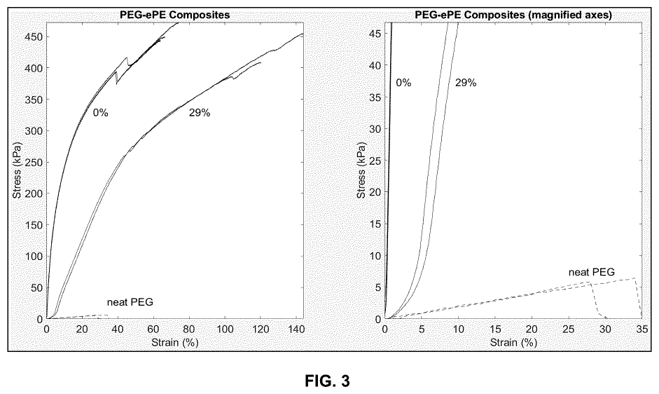

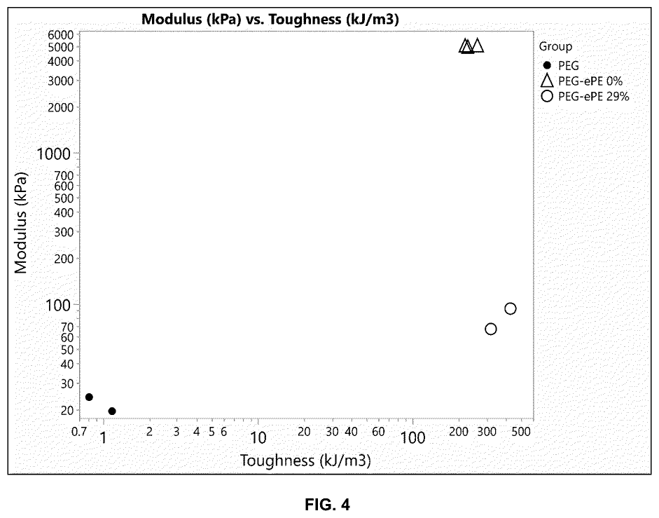

As in Example 1, the stress-strain curves (FIG. 3) and quantification of them (FIG. 4) demonstrate that while all composites were stronger and tougher than the unreinforced gel, only composites that were subjected to an in-plane compression prior to imbibing and curing maintained a low-strain modulus comparable to that of the gel. Mean (±std) values from FIG. 4 are recorded in Table 4.

TABLE 4
Silicone stretch ratio, resulting degree of in-plane film compression,
and mechanical properties for the composites and the neat PEG gel

| | | | In-plane | | |
|---|---|---|---|---|---|
| | | Silicone | Compression | | |
| | | Stretch | (1 − 1/λ) | Modulus | Toughness |
| Sample ID | Description | Ratio, λ | (%) | (kPa) | (kJ/m³) |
| 5 | PEG-ePE | 1.0 (no | 0% (no | 5,000 ± 57 | 240 ± 23 |
| (comparative) | 0% | stretch) | compression) | | |
| 6 | PEG-ePE | 1.4 | 29% | 80 ± 18 | 370 ± 76 |
| | 29% | | | | |
| 7 | Neat PEG gel | N/A | N/A | 22 ± 3.3 | 0.97 ± 0.23 |

### Example 3

The methodology of Example 1 (PTFE reinforced PEG hydrogel) was followed except that the PTFE membrane is used to reinforce a polyacrylamide (PAAm) hydrogel. We again demonstrate the ability to do so without substantially altering the modulus of the gel. One level of in-plane film compression is used (23%) and compared relative to the case where the reinforcing film is not compressed (0%) prior to imbibing and curing the hydrogel.

Film compression and handling as well as the mechanical characterization and analysis were conducted in a manner identical to that outlined in Example 1.

### Hydrogel Precursor Solution

Acrylamide (CAS 79-06-1; Sigma Aldrich, supra), N′N′-Methylenebis(acrylamide) (CAS 110-26-9; Sigma-Aldrich, supra), and ammonium persulfate (CAS 7727-54-0; Sigma-Aldrich, supra) were used to prepare the hydrogel precursor solution. A 25-mL solution of 10 wt. % acrylamide and 0.3 wt. % N′N′-Methylenebis(acrylamide) was prepared with DI water and degassed with N₂. Subsequently, 250 μL of 10 wt. % ammonium persulfate was added and degassed. This solution was made immediately prior to imbibing and curing. Hydrogel was formed based on the methods outlined in Sun et al., Nature, (2012), 489(7414): 133-136.

### Imbibing and Curing of the Hydrogel Precursor to Create Reinforced Gel Composites

Imbibing and curing was identical to that described in Example 1 except that the frame was first wet-out with isopropyl alcohol (IPA) and exchanged with water before quickly sandwiching, injecting the precursor, and curing the composite at 50° C. for at least 5 hrs.

### Results

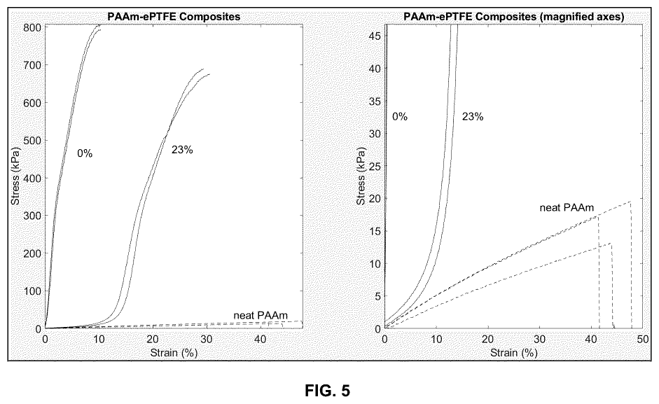

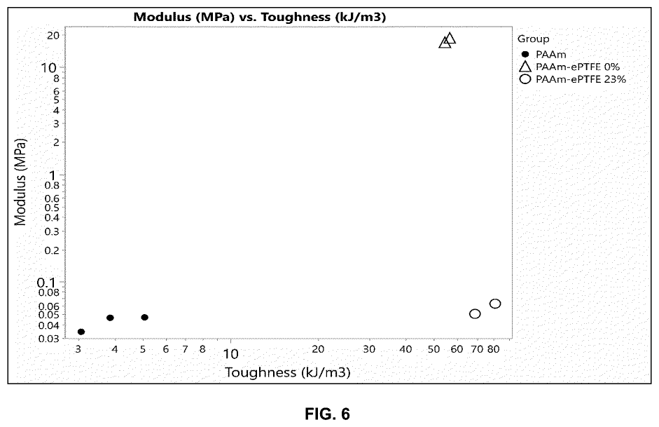

As in Example 1, the stress-strain curves (FIG. 5) and quantification of them (FIG. 6) demonstrate that while all composites were stronger and tougher than the unreinforced gel, only composites that were subjected to an in-plane compression prior to imbibing and curing maintained a low-strain modulus comparable to that of the gel. Mean (±std) values from FIG. 6 are recorded in Table 5.

TABLE 5
Silicone stretch ratio, resulting degree of in-plane film compression,
and mechanical properties for the composites and the neat PAAm gel

| | | | In-plane | | |
|---|---|---|---|---|---|
| | | Silicone | Compression | | |
| | | Stretch | (1-1/λ) | Modulus | Toughness |
| Sample ID | Description | Ratio, λ | (%) | (kPa) | (kJ/m³) |
| 8 | PAAm- | 1.0 (no | 0% (no | 18,000 ± 1,200 | 55 ± 1.5 |
| (comparative) | ePTFE 0% | stretch) | compression) | | |
| 9 | PAAm- | 1.3 | 23% | 56 ± 8.7 | 75 ± 8.5 |
| | ePTFE 23% | | | | |
| 10 | Neat PAAm | N/A | N/A | 42 ± 7.0 | 4.0 ± 1.0 |
| | gel | | | | |

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. A reinforced hydrogel composite comprising:
a porous synthetic or naturally derived retracted membrane material having a void volume, and a hydrogel at least partially filling the void volume;
wherein the composite has a low strain (<50%) modulus from about 0.01 to about 10 MPa and a toughness from about 10⁴ to about 10⁷ J·m³.

2. The reinforced hydrogel composite of claim 1, wherein the membrane has a microstructure, optionally comprising serpentine fibrils or a node and fibril structure.

3. The reinforced hydrogel composite of claim 2, wherein the fibrils in the node and fibril microstructure have an average diameter from about 0.1 μm to 250 μm.

4. The reinforced hydrogel composite of claim 2, wherein the nodes in the node and fibril microstructure have an average separation distance of about 5 to 5000 μm.

5. The reinforced hydrogel composite of claim 1, wherein the membrane comprises macro-structured folds or micro-structured folded fibrils.

6. The reinforced hydrogel composite of claim 1, wherein the membrane material is selected from the group consisting of expanded polytetrafluoroethylene (ePTFE), polyvinylidene fluoride (PVDF), fluorinated ethylene propylene (FEP), perfluoroalkoxy alkane (PFA), polyester sulfone (PES), expanded poly paraxylylene (ePPX), expanded ultra-high molecular weight polyethylene (eUHMWPE), expanded ethylene tetrafluoroethylene (eETFE), porous poly (tetramethyl-p-silphenylenesiloxane), expanded porous polylactic acid (ePLLA), polycaprolactone (PCL), polyurethane (PU), copolymers of polyglycolic acid (PGA) and trimethylene carbonate (TMC), silk fibroin, silk spidroin, cellulose, nanocellulose, polyhydroxyalkanoates, and any combination thereof.

7. The reinforced hydrogel composite of claim 1, wherein the hydrogel is a polyampholyte hydrogel or a zwitterionic hydrogel.

8. The reinforced hydrogel composite of claim 7, wherein the polyampholyte hydrogel comprises at least one cationic group and at least one anionic group.

9. The reinforced hydrogel composite of claim 8, wherein said cationic and anionic groups are randomly dispersed.

10. The reinforced hydrogel composite of claim 1, wherein the hydrogel comprises polyethylene glycol (PEG), polyvinyl alcohol (PVA), poly(2-hydroxyethyl methacrylate) (pHEMA), alginate, hyaluronic acid, or chitosan.

11. The reinforced hydrogel composite of claim 1, wherein the hydrogel is cross-linked.

12. The reinforced hydrogel composite of claim 1, wherein the hydrogel is cross-linked in situ.

13. The reinforced hydrogel composite of claim 1, wherein the hydrogel completely fills the void volume.

14. The reinforced hydrogel composite of claim 1, wherein the hydrogel further comprises one or more neutral groups.

15. The reinforced hydrogel composite of claim 1, wherein the reinforced hydrogel composite contains from 0.01 wt. % to 99 wt. % of the hydrogel based on the total weight of the reinforced hydrogel composite.

16. The reinforced hydrogel composite of claim 1, further comprising at least one bioactive agent.

17. The reinforced hydrogel composite of claim 16, wherein the bioactive agent is selected from the group consisting of thrombo-resistant agents, antibiotic agents, anti-tumor agents, anti-viral agents, anti-angiogenic agents, angiogenic agents, anti-inflammatory agents, cell cycle regulating agents, and chemically modified equivalents and combinations thereof.

18. The reinforced hydrogel composite of claim 1, wherein the polymer membrane is in the form of a tape, a sheet, a tube, a fiber, a filament, or a monolith.

19. The reinforced hydrogel composite of claim 18, wherein the tape, sheet, or tube has a thickness from about 1 μm to 5000 μm.

20. The reinforced hydrogel composite of claim 1, wherein the hydrogel is covalently bonded to the polymer membrane.

21. The reinforced hydrogel composite of claim 1, wherein the reinforced hydrogel composite contains from 10 to 95 volume percent of hydrogel within the void volume of the polymer membrane.

22. A laminate comprising the reinforced hydrogel composite of claim 1.

23. An article comprising the reinforced hydrogel composite of claim 1 or the laminate of claim 22.

24. The article of claim 23, wherein the article is an implantable medical device, a stent, a sensor, a fuel cell, a garment, footwear, a cosmetic, or a filter.

25. The article of claim 24, wherein the implantable medical device is selected from the group consisting of conduits, vascular grafts, endovascular grafts, stents, graft-stents, catheters, guidewires, trocars, tissue scaffolds, and introducer sheaths.

26. A method of preparing a reinforced hydrogel composite comprising:
i) providing a synthetic or naturally derived polymer membrane compressed in at least one direction, said polymer membrane having a node and fibril microstructure and a void volume;
ii) optionally pre-treating the polymer membrane;
iii) filling at least partially the void volume with a hydrogel precursor; and
iv) polymerizing the hydrogel precursor to produce the reinforced hydrogel composite which comprises a hydrogel embedded in the polymer membrane;
wherein the composite has low strain (<50%) modulus from about 0.01 to about 10 MPa and a toughness of from about 10⁴ to about 10⁷ J·m³.

27. The method of claim 26, wherein the pre-treating of the polymer membrane comprises wetting the polymer membrane with a solvent.

28. A method of claim 27, wherein the solvent is isopropyl alcohol or acetone.

29. The method of claim 26, wherein the pre-treating of the polymer membrane comprises wetting the polymer membrane with a hydrogel precursor solution.

30. The method of claim 29, wherein hydrogel precursor solution is UV-curable, chemically crosslinked, or physically crosslinked.

31. The method of claim 26, wherein the polymer membrane is a hydrophilic-treated membrane.

32. The reinforced hydrogel composite of claim 7, wherein the zwitterionic hydrogel includes poly(sulfobetaine methacrylate) (SBMA), poly(carboxybetaine methacrylate (CBMA), poly(2-methacryloyloxyethyl phosphorylcholine) (MPC), or carboxybetaine acrylamide (CBAA).
